(19) United States
(12) Patent Application Publication
(10) Pub. No.: US 2023/0398247 A1
(43) Pub. Date: Dec. 14, 2023

(54) STERILISATION APPARATUS FOR GENERATING HYDROXYL RADICALS

(71) Applicant: CREO MEDICAL LIMITED, Chepstow, Monmouthshire (GB)

(72) Inventors: Christopher Paul HANCOCK, Chepstow (GB); Malcolm WHITE, Chepstow (GB)

(21) Appl. No.: 18/033,945

(22) PCT Filed: Oct. 19, 2021
(86) PCT No.: PCT/EP2021/078969

§ 371 Date: Apr. 26, 2023

(30) Foreign Application Priority Data
Nov. 23, 2020 (GB) 2018344.8

(51) Int. Cl. A61L 2/14

(52) U.S. Cl. CPC A61L 2/14; A61L 2202/11; A61L 2202/15; A61L 2202/25

(57) ABSTRACT

Sterilisation apparatus suitable for generating hydroxyl radicals for sterilising an enclosed space, in which energy and water mist feeds are combined in a manner that permit the apparatus to be readily scaled to the size of enclosure. In particular, the sterilisation apparatus provides a manifold for providing a plasma generating region to form a plasma arc through which a flow of water mist is directed to form the hydroxyl radicals. A power distribution device transmits microwave energy generated by a microwave source to the manifold and distributes the received microwave energy to a plurality of output ports connected to the manifold.

### FIELD OF THE INVENTION

The invention relates to sterilisation systems suitable for clinical use, e.g. on the human body, medical apparatuses, or hospital bed spaces. For example, the invention may provide a system that can be used to destroy or treat certain bacteria and/or viruses associated with the human or animal biological system and/or the surrounding environment. This invention is particularly useful for sterilising or decontaminating enclosed or partially enclosed spaces.

### BACKGROUND TO THE INVENTION

Bacteria are single-celled organisms that are found almost everywhere, exist in large numbers and are capable of dividing and multiplying rapidly. Most bacteria are harmless, but there are three harmful groups; namely: cocci, spirilla, and bacilla. The cocci bacteria are round cells, the spirilla bacteria are coil-shaped cells, and the bacilli bacteria are rod-shaped. The harmful bacteria cause diseases such as tetanus and typhoid.

Viruses can only live and multiply by taking over other cells, i.e. they cannot survive on their own. Viruses cause diseases such as colds, flu, mumps and AIDS. Viruses may be transferred through person-to-person contact, or through contact with region that is contaminated with respiratory droplets or other virus-carrying bodily fluids from an infected person.

Fungal spores and tiny organisms called protozoa can cause illness.

Sterilisation is an act or process that destroys or eliminates all form of life, especially micro-organisms. During the process of plasma sterilisation, active agents are produced. These active agents are high intensity ultraviolet photons and free radicals, which are atoms or assemblies of atoms with chemically unpaired electrons. An attractive feature of plasma sterilisation is that it is possible to achieve sterilisation at relatively low temperatures, such as body temperature. Plasma sterilisation also has the benefit that it is safe to the operator and the patient.

Plasma typically contains charged electrons and ions as well as chemically active species, such as ozone, nitrous oxides, and hydroxyl radicals. Hydroxyl radicals are far more effective at oxidizing pollutants in the air than ozone and are several times more germicidal and fungicidal than chlorine, which makes them a very interesting candidate for destroying bacteria or viruses and for performing effective decontamination of objects contained within enclosed spaces, e.g. objects or items associated with a hospital environment.

OH radicals held within a “macromolecule” of water (e.g. a droplet within a mist or fog) are stable for several seconds and they are 1000 times more effective than conventional disinfectants at comparable concentrations.

An article by Bai et al titled “Experimental studies on elimination of microbial contamination by hydroxyl radicals produced by strong ionisation discharge” (Plasma Science and Technology, vol. 10, no. 4, August 2008) considers the use of OH radicals produced by strong ionisation discharges to eliminate microbial contamination. In this study, the sterilisation effect on E. coli and B. subtilis is considered. The bacteria suspension with a concentration of 10⁷ cfu/ml (cfu=colony forming unit) was prepared and a micropipette was used to transfer 10 μl of the bacteria in fluid form onto 12 mm×12 mm sterile stainless steel plates. The bacteria fluid was spread evenly on the plates and allowed to dry for 90 minutes. The plates were then put into a sterile glass dish and OH radicals with a constant concentration were sprayed onto the plates. The outcomes from this experimental study were:
1. OH radicals can be used to cause irreversible damage to cells and ultimately kill them;
2. The threshold potential for eliminating micro-organisms is ten thousandths of the disinfectants used at home or abroad;
3. The biochemical reaction with OH is a free radical reaction and the biochemical reaction time for eliminating micro-organisms is about 1 second, which meets the need for rapid elimination of microbial contamination, and the lethal time is about one thousandth of that for current domestic and international disinfectants;
4. The lethal density of OH is about one thousandths of the spray density for other disinfectants—this will be helpful for eliminating microbial contamination efficiently and rapidly in large spaces, e.g. bed-space areas; and
5. The OH mist or fog drops oxidize the bacteria into CO₂, H₂O and micro-inorganic salts. The remaining OH will also decompose into H₂O and O₂, thus this method will eliminate microbial contamination without pollution.

WO 2009/060214 discloses sterilisation apparatus arranged controllably to generate and emit hydroxyl radicals. The apparatus includes an applicator which receives RF or microwave energy, gas and water mist in a hydroxyl radical generating region. The impedance at the hydroxyl radical generating region is controlled to be high to promote creation of an ionisation discharge which in turn generates hydroxyl radicals when water mist is present. The applicator may be a coaxial assembly or waveguide. A dynamic tuning mechanism e.g. integrated in the applicator may control the impedance at the hydroxyl radical generating region. The delivery means for the mist, gas and/or energy can be integrated with each other.

WO 2019/175063 discloses a sterilisation apparatus that uses thermal or non-thermal plasma to sterilise or disinfect surgical scoping devices. In one example, a plasma generating region is formed at a distal end of a coaxial transmission line, which convey RF or microwave energy to strike and sustain the plasma. A gas passageway is formed around an outer surface of the coaxial transmission line. The gas passageway is in fluid communication with the plasma generating region through notches in a cylindrical electrode mounted on a distal end of the coaxial transmission line. In some examples, water through a passageway formed within the inner conductor of the coaxial transmission line, from where it is sprayed on to the surface of an object before the plasma passes over it.

### SUMMARY OF THE INVENTION

At its most general, the invention provides a sterilisation apparatus suitable for generating hydroxyl radicals for sterilising an enclosed space, in which energy and water mist feeds are combined in a manner that permit the apparatus to be readily scaled to the size of enclosure. In particular, the sterilisation apparatus provides a manifold for providing a plasma generating region to form a plasma arc through which a flow of water mist is directed to form the hydroxyl radicals. A power distribution device transmits microwave energy generated by a microwave source to the manifold and distributes the received microwave energy to a plurality of output ports connected to the manifold.

According to one aspect of the invention, there is provided a sterilisation apparatus as set out in claim 1. The sterilisation apparatus comprises a microwave source arranged to generate microwave energy, a mist generator arranged to generate a flow of water mist, a manifold, and a power distribution device. The manifold is connected to receive the flow of water mist from the mist generator and defines a plasma generating region. The power distribution device is configured to transmit microwave energy, in particular from the microwave source, to the manifold. The power distribution device includes an input port coupled to the microwave source and a plurality of output ports. The plurality of output ports leads to the plasma generating region. The manifold is configured to direct the flow of water mist through the plasma generating region to a manifold outlet. The power distribution device is configured to split microwave energy received at the input port between the plurality of output ports. For example, the power distribution device may include or may operate as a power splitter that divides power at the input port between the plurality of output ports.

An advantage of the invention is that the microwave energy generated by the microwave source and received by the power distribution device at the input port is distributed to the plurality of output ports. Therefore, it is possible to apply the microwave energy at several locations spaced apart from each other. This helps to create a plasma arc which extends over a sufficiently large volume. The higher the volume of the plasma generating region is the more hydroxyl radicals can be generated which increases the sterilisation capabilities of the sterilisation apparatus. The power distribution device provides a simple and effective means for distributing the microwave energy over the plasma generating region.

Furthermore, the power distribution device may provide a direct connection between the microwave source and the manifold such that cables, for example coaxial cables, for transmitting the microwave energy can be completely omitted. This may reduce the complexity of the sterilisation apparatus and minimises transmission losses. In one example, the power distribution device may be a waveguide-based power splitter coupled directly between the microwave source and the manifold.

The sterilisation apparatus may be configured as a device or means for sterilising volumes (such as rooms) and/or surfaces by generating and applying hydroxyl radicals. The hydroxyl radicals may be emitted from the sterilisation apparatus at a manifold outlet of the manifold.

The manifold may include a manifold inlet which is configured to receive water mist from the mist generator. The mist may be passed through the manifold to the manifold outlet at which the mist exits the manifold. On the way from the manifold inlet to the manifold outlet, the mist passes the plasma generating region where a plasma is present which generates the hydroxyl radicals. Thus, the mist exiting the manifold outlet includes the hydroxyl radicals which provide the sterilisation capabilities of the sterilisation apparatus.

The manifold may comprise a hollow body that acts as a fluid flow conduit from the manifold inlet to the manifold outlet. For example, the manifold may define a flow direction of the water mist from the manifold inlet to the manifold outlet. The flow direction may be aligned with the direction of the flow of water mist that is received into the manifold. That is, the water mist is substantially undeflected as it travels through the plasma generating region. This may be advantageous in obtaining a large sterilisation range for a given water mist flow rate.

The plasma generating region may be arranged between the manifold inlet and the manifold outlet. The manifold may include one or more plasma cavities in which the plasma is generated. The plasma generating region may be a part of the plasma cavity, in particular that part of the volume of the plasma cavity in which the plasma is generated. In other words, the body of the manifold includes one or more plasma cavities. The plasma cavities may be in fluid communication with each other or are separated from each other. The plasma cavities may not be in fluid communication with each other.

At least one output port is connected to each plasma cavity in order to supply microwave energy to the plasma cavity for striking and/or maintaining the plasma.

The manifold may comprise a plurality of lateral ports configured to connect with the output ports of the power distribution device. The position and orientation of the lateral ports may define the plasma generating region, i.e. where the plasma is located. The lateral ports may be configured to be directly connected to the output ports. Alternatively or additionally, the lateral ports may be configured to receive plasma applicators to be described later.

At least one lateral port is arranged with each plasma cavity. The lateral port is arranged on a side of the plasma cavity. The position and/or orientation of the one or more lateral ports (and, thus, of the output ports) defines the area where the plasma is generated or, in other words, the plasma generating region.

The manifold may be made (e.g. moulded) from an electrically insulating material so that it does not interfere with the delivery of the microwave energy.

The manifold inlet and/or the manifold outlet may be detachable from the body. Thus, it is possible to adjust the manifold inlet and/or the manifold outlet in view of the number of mist generators and/or the size or shape of the enclosure.

The manifold inlet and/or the manifold outlet are in fluid communication with the one or more plasma cavities.

The manifold inlet may be provided to establish a homogeneous flow of the water mist through the plasma generating region. For example, the manifold inlet is configured to combine several incoming flows of water mist to the plasma cavity or to distribute the one or more incoming flows of water mist to a plurality of plasma cavities.

The manifold outlet may be provided to guide the flow of water mist to the enclosure. For example, the manifold outlet is configured to combine the flows of water mist from the plasma cavities.

The microwave source may be generator capable of producing microwave energy having a power suitable for striking and/or maintaining a plasma. The microwave source may be configured to generate microwave radiation. In one example, the microwave source comprises a magnetron. In other examples, the microwave source may comprise an oscillator and a power amplifier. The microwave source may include solely one source outlet for outputting the microwave energy generated by the microwave source. For example, the microwave source may include one opening for emitting the microwave energy in form of radiation. This opening may be connected to the input port of the power distribution device.

The microwave source may be configured to generate microwave energy of a single frequency, generate microwave energy of a particular bandwidth of frequencies, or selectively generate microwave energy of different frequencies. For example, a first frequency of microwave energy is generated to strike a plasma and a second frequency of microwave energy is generated to maintain the plasma.

The mist generator may comprise any suitable means for generating a mist of water droplets or water vapour. For example, the mist generator may be an ultrasonic fogging device in which ultrasonic vibrations are applied to a water source to generate fine water droplets. In another example, the mist generator may operate to heat water to produce water vapour.

The sterilisation apparatus may comprise a plurality of mist generators, wherein the manifold inlet comprises a plurality of manifold openings, each manifold opening being connectable to a respective mist generator. The apparatus may thus be scalable by adapting the manifold inlet to receive a desired number of mist generator inputs. This may be achieved by detachably connecting the manifold inlet which includes a number of openings corresponding to the number of mist generators.

In an optional embodiment, the sterilisation apparatus comprises a gas supply which is connected to deliver a gas flow to the mist generator, wherein preferably the gas flow entrains water mist formed by the mist generator to create the flow of water mist.

In this way, the flow rate of the mist may be controllable. This may be particularly desirable if there are a plurality of mist generators, where it may be useful to be able to independently control the gas flow rate for each mist generator, e.g. in order to ensure that a uniform flow is received within the manifold.

Preferably the gas supply is a supply of argon gas. However, any other suitable gas may be chosen, e.g. carbon dioxide, helium, nitrogen, a mixture of air and any one of these gases, for example 10% air/90% helium.

The sterilisation apparatus may be configured for use with an enclosure. For example, the manifold outlet may be couplable to an enclosure, such as a box, room, vehicle or the like. The enclosure may define a space to be sterilised. The apparatus may be scaled to the size of the enclosure. For example, the number of mist generators, the flow rate of gas, and the number of plasma applicators and all factors that can be adapted depending on the enclosure. By providing a manifold capable of combining inputs from multiple individual components, the apparatus of the invention facilitates the ability to adapt to different environments.

The power distribution device is a means for transmitting microwave energy from the microwave source to the manifold. The power distribution device may include a power splitter which can also be called a power divider. In particular, the power distribution device is a means for transmitting microwave energy in form of radiation.

In particular, the power distribution device provides a direct connection from the source outlet to lateral ports of the manifold. Further waveguides and/or (coaxial) cables are omitted for supplying the microwave energy form the microwave source to the manifold.

The power splitter is a device which is capable of distributing the power of incoming microwave energy, in particular incoming microwave radiation, to the plurality of output ports. Optionally, the power of the microwave energy or radiation is approximately the same at each output port. Preferably, the power splitter can function as a power combiner if the radiation is inputted into the output ports.

The power distribution device may be configured (e.g. have a geometry selected) to exhibit a low loss power splitting function for at least at the frequencies of the microwave energy which are generated by the microwave source. Thus, the power splitting capabilities may not be present in full at microwave frequencies different to the ones generated by the power source.

The input port of the power distribution device may be directly connected/coupled to the source outlet of the microwave source. The output ports of the power distribution device may be directly coupled to the lateral ports of the manifold.

The sterilisation apparatus may comprise two or more power distribution devices and/or two or more manifolds. Preferably, the two or more power distribution devices are connected to lateral ports of one manifold. Alternatively, each manifold is connected to the output ports of a single power distribution device. In this configuration, the microwave source may have two or more source outlets; each source outlet is connected to the input port of a respective power distribution device.

It is also possible that a single main power distribution device is connected to the single source outlet of the microwave source via its input port. The output ports of the main power distribution device are connected to the respective input ports of the two or more power distribution devices.

In use, the manifold receives a flow of water mist that is directed through a plasma generating region in which the plasma is generated by the microwave source. The mechanism for plasma generation is independent of the water mist delivery. Moreover, it permits the sterilisation apparatus to be scalable both in terms of the size of the plasma generating region (controlled by the number of plasma applicators or manifold inlets) and in terms of the flow rate (volume per second) of water mist. The manifold may be adapted to combine together water mist inputs from multiple mist generators as well as receiving a plurality of plasma applicators. The output ports of the power distribution device provides the microwave energy to the plasma generating region in order to strike and/or maintain the plasma within the plasma generating region.

The plasma may be struck directly by energy delivered from the power distribution device. That is, the plasma may be struck without requiring a separate device or means for creating a high voltage condition. For example, the manifold and/or output ports of power distribution device may be configured (i.e. have a geometry selected) to present an impedance (in the presence of water mist and absence of plasma) at the plasma generating region that causes an electric field having a strength capable of striking a plasma. The presence of the plasma changes the impedance in the plasma generation region. The impedance at the outputs of the power distribution device may be configured to match with the impedance of the plasma generation region when the plasma is preset. For example, the impedance at the outputs of the power distribution device may be 50Ω.

In some embodiments, the lateral ports of the manifold and/or the output ports may include means for locally increasing the electric field generated by the microwave radiation to such a level that the strike and/or the maintenance of the plasma is possible. For example, the means may include a tip and/or an edge of a conductive material which locally increases the electric potential of the structure and, therefore, the electric field generated by the microwave radiation. A conductive needle or other sharp devices are example of such means for increasing the local electric field generated by the microwave radiation.

In yet further embodiments, a separate device for striking the plasma may be provided. For example, one or more output ports, preferably not all output ports, may be provided with a plasma applicator as described below. The plasma applicators are capable of striking and maintaining the plasma.

In one example, the plasma may be struck by providing a radio frequency (RF) source configured to generate a RF pulse. The RF pulse is fed to the plasma generating region to strike the plasma. After the plasma is struck, the RF source is switched off as the plasma is maintained by the microwave energy, i.e. the microwave energy supplied by the microwave source provided the energy to maintain the plasma.

As mentioned above, the power distribution device, in particular the power splitter, comprises a waveguide, preferably an assembly of interconnected waveguides. Preferably, the power splitter and/or the power distribution device consist solely of waveguides. This means, the power splitter and/or the power distribution device are capable of receiving microwave radiation at the input port and/or of emitting the microwave radiation at the output ports. Thus, microwave radiation is supplied to the manifold. In other words, microwave radiation is received at the input port and distributed to the plurality of output ports.

The waveguides that are interconnected in the assembly are connected to each other and/or intersect each other at junctions. The junctions may be the location at which the incoming power in one waveguide is distributed to two or more waveguides. Thus, the junctions may facilitate a division or splitting of the power.

The positioning of the junction within the assembly of interconnected waveguides determines efficiency and ratio of the power distribution at the junction. For example, the power distribution depends on the length of the waveguide to the junction in relation to the wavelength of the microwave energy, as explained in more detail below.

The term “waveguide” is used herein to mean a structure for guiding microwave radiation having the form of an elongated chamber or passage along which the microwave radiation propagates. This elongated chamber or passage is surrounded by a conductive material.

In one embodiment, the waveguide is a hole in a block made from a conductive material or the waveguide includes a waveguide body made from a plastic material, wherein an inner surface of the waveguide body is covered by a conductive layer. An assembly of interconnected waveguides may be provided by a plurality of holes in a block. The plurality of holes are in fluid communication with each other forming junctions at the position where two holes intersect each other. The holes may be formed by drilling which provides a simple manufacturing method for manufacturing an assembly of interconnected waveguides.

The block may be made from a metal. The blocks may be a cuboid having a base area between 100 mm² and 200 mm² and a height of 60 mm to 120 mm. One optional embodiment provides a block having a base area of 167 mm² and a height of 90 mm².

Alternatively or additionally, the waveguide includes a waveguide body made from a plastic material whose inside surface is covered by a conductive layer. The conductive layer is required for the propagation of the microwave radiation with in the waveguide body. The thickness of the conductive layer is greater than the skin depth for the microwave radiation transmitted within the waveguide body. Preferably, the complete inner surface of the waveguide body is covered by the conductive layer. The material of the conductive layer may be a metal.

The advantage of this embodiment of the waveguide is the reduced weight due to the manufacturing of the waveguide body from a plastic material.

Alternatively, the waveguide body is completely made from a conductive material.

A junction for distributing the power may be formed in that an end of the waveguide body is connected to an opening in a side surface of another waveguide body forming a T-junction. Other types of junctions can be manufactured by connecting two or more waveguides bodies to each other.

In one example, the power splitter may include a ring coupler, wherein preferably the output port of the ring coupler is orientated radially inwards.

The ring coupler may include a ring transmission line which has the shape of a ring. Radial transmission lines are connected to the ring transmission line and protrude radially from the ring transmission line. The radial transmission lines can define the input port and/or the plurality of output ports. The length of the radial transmission line (i.e. the length between the end of the radial transmission line and its connection point to the ring transmission line) can have a particular length which is chosen to provide a good transmission ratio from or to the ring transmission line. For example, the length of the radial transmission line may be a half of the wavelength of the frequency of the microwave energy.

The ring transmission line and/or the radial transmission line may constituted by waveguides.

In one embodiment, the radial transmission lines defining the output ports protrude radially inwards from the ring transmission line. The plasma generating region is optionally surrounded by the ring transmission line. The output ports are connected to a radially outward facing surface of the body defining the plasma cavity.

The provision of radially inward extending radial transmission lines and, therefore, output ports arranged radially inwards of the ring transmission line provides an arrangement in which the microwave energy is fed into the plasma generating region having a circle structure. In particular, it is possible that the output ports (and therefore the corresponding lateral ports of the manifold) are equally distributed in a circumferential direction around the plasma generating region. This can facilitate the generation of a homogeneous plasma generating region.

In an optional embodiment, the radial transmission lines protrude radially outwards from the ring transmission line. In this embodiment, the plasma cavity and, preferably, the plasma generating region has the shape of a torus which extends along the ring transmission line. Preferably, the ring transmission line and the plasma cavity are coaxially arranged. The output ports are connected to a radially inward facing surface of the body defining the plasma cavity.

In an optional embodiment, the distance between any two output ports corresponds to nλ/2, wherein n is an integer and λ is the wavelength of the microwave energy in the waveguide. The input port of the ring coupler is preferably disposed between and at an equal distance from two output ports. With this arrangement, the distance between two radial transmission lines each defining the output port along the extension of the ring transmission line corresponds to a multitude of half the wavelength of the microwave energy. For example, any two output ports are spaced apart by half of the wavelength. However, it is possible that the distance between two output ports varies: for example the distance between two output ports is half a wavelength while the distance between other two output ports is a multiple of half the wavelength.

The radial transmission line defining the input port is preferably arranged exactly in the middle between two radial transmission lines connected to respective output ports. In other words, the input port has a distance from an output port in the extension of the ring transmission line of λ/4 (or any other odd number of quarter wavelengths).

The above-described distances increase the coupling/transmission of the microwave energy from the ring transmission line to the radial transmission line and vice versa. In addition, this embodiment facilitates an equal distribution of the power of the microwave energy over the outlets.

In an optional embodiment, the power splitter comprises a plurality of interconnected straight waveguides that provide a plurality of paths from the input port to the outlet ports, where each path comprises a plurality of orthogonally disposed waveguide sections that interconnect junctions between the waveguides. The power splitter may be configured such that each junction is distanced from a previous junction or the input port by nλ/2, wherein n is an integer and λ is the wavelength of the microwave energy as it propagates through the waveguide.

The straight waveguides or an assembly of straight waveguides may be constituted by the above described holes in a block made from a conductive material or by the above described waveguide body (waveguide bodies) made from a plastic material whose inner surfaces is covered with a conductive layer.

The distance between the input port or a junction to another junction of a multiple of half a wavelength of the microwave energy has been shown to provide a good transmission ratio, i.e. the backscattering at the junction can be minimised. Furthermore, this facilitates an equal distribution of the power at the junction.

In an optional embodiment, the power splitter comprises a Wilkinson power divider.

For example, the Wilkinson power divider may be additionally used to the above described embodiments of the power splitter. Alternatively, the power distribution device includes a power splitter including Wilkinson power dividers and another power splitter made of other embodiments described herein.

The Wilkinson power divider may be employed if the impedance of the power distribution device is supposed to have 100Ω. The other described embodiments of the power splitter are preferably employed for achieving an impedance of the power distribution device of 50Ω. The plasma has an impedance of approximately 50Ω such that there is good impedance match with the power distribution device resulting in a good transmission of energy into the plasma.

The power distribution device may include a plurality of power splitters. For example, the power distribution device may include a plurality of ring couplers which are spaced apart in the direction of the flow of the mist. In this case, each ring coupler may provide a local homogeneous plasma which is extended in the direction of the flow of the mist by the additional ring coupler(s).

As mentioned above, in one example, the sterilisation apparatus may include a plurality of plasma applicators which are connected to the output ports. For example, the plasma applicators are positioned in or at the lateral ports of the manifold. In particular, one plasma applicator is positioned in or at one lateral port of the manifold. The lateral ports of the manifold can be configured to support the plasma applicators. Preferably, the output ports are connected to the plasma applicators.

Each plasma applicator may extend transversely to the flow of water mist through the plasma generating region. For example, the manifold may comprise a plurality of lateral ports (i.e. ports in a side surface thereof) to receive the plasma applicators. With this arrangement, the direction in which energy is injected into the plasma generating region may thus be orthogonal to the flow of water mist.

More generally, the orientation of the lateral ports which can be constituted by waveguides or openings in the waveguide body can be orthogonal to the flow of water mist.

The plurality of plasma applicators may comprise one or more pairs of plasma applicators that face one another on opposing sides of the plasma generating region. The plasma generating region may comprise or consist of a space between the one or more pairs of plasma applicators.

The plurality of plasma applicators may be arranged around the plasma generating region in a manner that causes their respective plasma arcs to combine to form a ring.

Each plasma applicator may be configured to strike a plasma using the microwave energy only. However, in other embodiments the apparatus may include an RF source arranged to supply a pulse of RF energy to strike the plasma, with the microwave energy used to sustain it. An example of an RF strike and microwave sustain set up is given in WO 2019/175063.

In an arrangement capable of striking the plasma using microwave energy only, each plasma applicator may comprise: a conductive tube; and an elongate conductive member extending along a longitudinal axis of the conductive tube. The conductive tube and elongate conductive member may provide a first coaxial transmission line at a proximal end of the plasma applicator, and a second coaxial transmission line at a distal end of the plasma applicator. The first coaxial transmission line may be configured as a quarter wavelength impedance transformer. The quarter wavelength impedance transform may operate to transform a first impedance (e.g. of a coaxial cable that feeds the plasma applicator) to a second impedance (e.g. the impedance of the second coaxial transmission line). The second coaxial transmission line may be configured with a higher impedance than the first coaxial transmission line. An impedance of the first and second coaxial transmission lines may be determined by the geometry of the structure, e.g. the relative size of the diameter of the elongate conductive member and the inner diameter of the conductive tube. The second coaxial transmission line may have an impedance selected to establish an electric field at its distal end that is suitable to strike a plasma in the gas that flows through the plasma applicator. The flow of gas received by each plasma applicator may pass between the conductive tube and the elongate conductive member, where it also acts as a dielectric (insulating) material of the first and second coaxial transmission lines.

A sleeve of insulating material, e.g. quartz or the like, may be mounted in a distal end of the conductive tube. The sleeve may assist in focussing the electric field at the distal end of the second coaxial transmission line, thereby facilitating the plasma strike at a desired location.

Each plasma applicator may comprise a gas inlet tube configured to deliver the flow of gas to a space between the conductive tube and the elongate conductive member. The gas inlet tube may extend transversely to the longitudinal axis of the conductive tube.

In an optional embodiment, an adapter for connecting the output port to the plasma applicator is provided.

Each plasma applicator may comprise a proximal adapter configured to connect to the output port of the power distribution device. The proximal adapter may be configured feed the microwave radiation to the elongate conductive member and/or to the conductive tube. The microwave energy may thus be delivered in line with the longitudinal axis of the conductive tube, which may assist in efficient coupling. Meanwhile, the gas inlet tube may be arranged transversely to the longitudinal axis, which may be advantageous because it does not interfere with the delivery of the microwave energy.

The adapter may function as a wave to coaxial adapter. It is possible that the plasma applicator has a connector configured to connect to a coaxial cable and the adapter is a wave to coaxial adapter. In this case, the connector of the plasma applicator is directly attached to the adapter.

Optionally, one plasma applicator is provided with each lateral port of the manifold and/or one plasma applicator is connected to each output port of the power distribution device.

In a preferred embodiment, one or all plasma applicators can be omitted. In this case, an adapter arranged at the output port having a waveguide structure can be omitted, too. This means, the microwave radiation distributed by the power distribution device is directly fed into the plasma generating region. There, the microwave radiation strikes and/or maintains the plasma. The output ports of the power distribution device may emit microwave radiation and the lateral ports of the manifolds are configured as waveguides or openings allowing the transmission the microwave radiation into the plasma generating region. The orientation of the lateral ports may be the same as the orientation of the plasma applicators. This embodiment has the advantage that transmission losses at the interface between the waveguide and the adapter and/or the plasma applicator are not present.

In an embodiment in which the adapter is a waveguide to coaxial adapter, the power distribution device includes waveguides which end at the output ports. In this embodiment, the power is distributed by using waveguides while the microwave energy supplied to the manifold is output as electrical energy.

In an optional embodiment, the plurality of output ports is disposed around the plasma generating region.

For example, the ring coupler described above is an embodiment of an arrangement at which the plurality of output ports is disposed around the plasma generating region. The output ports may be arranged equally distributed around the plasma generating region. It is also possible that the output ports are arranged at one or more sections of the perimeter of the plasma generating region. Furthermore, the plurality of output ports may be arranged on one or more sides of the plasma generating region. For example, the output ports may be arranged at opposing sides of the plasma generating region.

In other words, the output ports are arranged radially outside the plasma generating region when viewed along the flow of water mist. The power distribution device may cover parts of the manifold. The lateral ports of the manifold are arranged on an outer surface of the manifold.

In an alternative embodiment, the plasma generating region has a torus shape.

In this embodiment, parts of the power distribution device are arranged radially inside of the torus defined by the plasma generating region. In this embodiment, the body of the manifold may include a plasma cavity having the shape of a torus. The lateral ports may be arranged on a radially inner surface of the body of the manifold. The output ports may extend radially outwards when viewed along the direction of the flow of the water mist.

The manifold inlet and the manifold outlet can be arranged on axial sides of the torus i.e. on sides when viewed in a direction of the flow of mist or opposite thereto. The flow of water mist may therefore form a hollow cylinder when travelling through the plasma cavity. The ring coupler may be used in conjunction with this embodiment of the manifold. However, it is also possible that the above described block including holes may be arranged within the manifold in order to provide output ports that extend radially outward.

The manifold inlet and/or the manifold outlet may have the outer shape of a funnel. The manifold inlet may be configured to distribute the flow of water mist from a single inlet to the toroid plasma generating region. Conversely, the manifold outlet may be configured to channel the flow of water mist from the toroid plasma generating region to a single aperture of the manifold outlet.

In a further alternative embodiment, the manifold comprises a first portion defining a first plasma generating region and a second portion defining a second plasma generating region, wherein preferably the power distribution device is arranged between the first portion and the second portion.

The first portion may be a part of the body of the manifold and the second body may be another part of the body of the manifold. The first portion and the second portion may be connected to each other or can be separately supported. The first portion may define a first plasma cavity and the second portion may define a second plasma cavity. The first plasma cavity and the second plasma cavity may not be in fluid communication with each other. For example, at least a part of the power distribution device is sandwiched between the first portion and the second portion. The lateral ports may be arranged on one side of the first portion and/or the second portion, wherein this side faces the power transmission device. In other words, the side at which the lateral ports are arranged is a radially inward side of the first portion and/or the second portion.

The power splitter used with this embodiment of the manifold may be the above described block. In this case, the output ports are arranged on a first side and a second side wherein the first site and the second side are opposite sides, for example an upper side and a lower side.

The manifold inlet may include a one or more openings in an axial side surface in each of the first portion and the second portion. The manifold outlet may be a one or more apertures in the other axial side surface in each of the first portion and the second portion.

Herein, the term “inner” means radially closer to the centre (e.g. axis) of the coaxial cable, probe tip, and/or applicator. The term “outer” means radially further from the centre (axis) of the coaxial cable, probe tip, and/or applicator.

The term “conductive” is used here to mean electrically conductive, unless the context dictates otherwise.

Herein, the terms “proximal” and “distal” refers to the ends of the applicator. In use, the proximal end is closer to a generator for providing the RF and/or microwave energy, whereas the distal end is further from the generator.

In this specification “microwave” may be used broadly to indicated a frequency range of 400 MHz to 100 GHz, but preferably in the range 1 GHz to 60 GHz. Specific frequencies that have been considered are: 915 MHz, 2.45 GHz, 3.3 GHz, 5.8 GHz, 10 GHz, 14.5 GHz, and 25 GHz. In contrast, this specification uses “radiofrequency” or “RF” to indicate a frequency range that is at least three orders of magnitude lower, e.g. up to 300 MHz, preferably 10 kHz to 1 MHz, and most preferably 400 kHz. The microwave frequency may be adjusted to enable the microwave energy delivered to be optimised. For example, a probe tip may be designed to operate at a certain frequency (e.g. 900 MHz), but in use the most efficient frequency may be different (e.g. 866 MHz).

### BRIEF DESCRIPTION OF THE DRAWINGS

Features of the invention are now explained in the detailed description of examples of the invention given below with reference to the accompanying drawings, in which:

FIG. 1 is a schematic diagram of a sterilisation apparatus according to an embodiment of the present invention;

FIG. 2 is a schematic cross-sectional view of a microwave source, a manifold and a power distribution device of the sterilisation apparatus of FIG. 1;

FIG. 3 is a schematic side view of an embodiment of the power distribution device;

FIG. 4 is a schematic side view of another embodiment of the power distribution device;

FIG. 5 is a schematic diagram of a sterilisation apparatus according to another embodiment of the present invention;

FIG. 6 is a schematic cross-sectional view of the microwave source, the manifold and the power distribution device of the sterilisation apparatus of FIG. 5;

FIG. 7 is a schematic diagram of a sterilisation apparatus according to a further embodiment of the present invention;

FIG. 8 is a schematic cross-sectional view of the microwave source, the manifold and the power distribution device of the sterilisation apparatus of FIG. 7;

FIG. 9 is a schematic side view of an embodiment of the power distribution device;

FIG. 10 is a schematic side view of another embodiment of the power distribution device;

FIG. 11 is a schematic top view of a feed manifold suitable for use with the sterilisation apparatus of FIG. 1;

FIG. 12 is a schematic cross-sectional view of the microwave source, the manifold, the power distribution device and plasma applicators suitable for use in the sterilisation apparatus of FIG. 1;

FIG. 13 is a schematic side view of the plasma applicator depicted in FIG. 12; and

FIG. 14 is a schematic cross-sectional view of the plasma applicator of FIG. 13.

### DETAILED DESCRIPTION; FURTHER OPTIONS AND PREFERENCES

This invention relates to a device for performing sterilisation using hydroxyl radicals that are generated by creating a plasma in the presence of water mist.

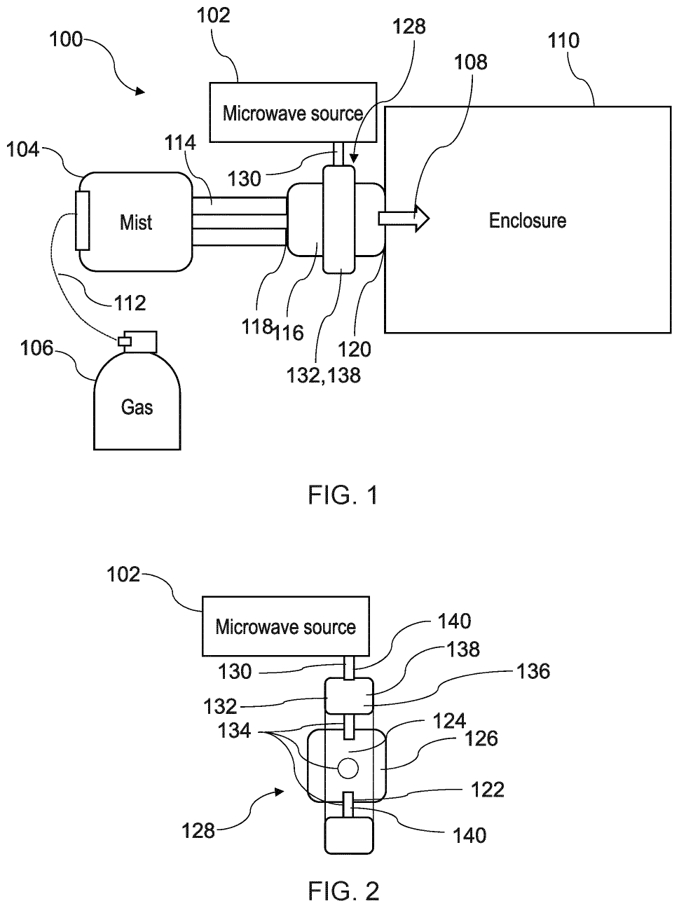

FIG. 1 is a schematic view of a sterilisation apparatus 100 that is an embodiment of the invention. The sterilisation apparatus 100 operates to combine feeds from each of a microwave source 102, a mist generator 104 and a gas supply 106 to generate a flow 108 of hydroxyl radicals into an enclosure 110 to be sterilised.

The microwave source 102 may be any suitable microwave generator for outputting microwave energy, i.e. electromagnetic energy having a frequency in a range of 400 MHz to 100 GHz, preferably in the range 1 GHz to 60 GHz. For example, it may be a magnetron arranged output microwave radiation having a frequency of 2.45 GHz. In other embodiments, the microwave source 102 may comprise an oscillator and power amplifier. The microwave source 102 may be configured to output microwave energy with a power equal to or greater than 200 W, preferably 500 W or more, e.g. 800 W or the like. The microwave source 102 is configured to emit microwave radiation at a source outlet.

The mist generator 104 may comprise one or more ultrasonic fogging devices, in which a fine mist of water droplets is obtained by applying ultrasonic energy to a vessel storing liquid water, e.g. distilled water. Alternatively, the mist generator 104 may comprise a device for generating water vapour (steam) by applying heat to stored water.

The gas supply 106 may comprise a canister of pressurised inert gas, such as argon, nitrogen, carbon dioxide or the like. Alternatively, the sterilisation apparatus may operate with air as the gas medium in which a plasma is struck. In this example, the gas supply 106 may comprise a fan or other means for generating a directable gas flow.

In this example, the gas supply 106 has a connection 112 through which a gas flow is supplied to the mist generator 104. The gas flow entrains the mist or water vapour from the mist generator 104 and conveys it through mist conduits 114 towards the enclosure 110. Where there are multiple mist generators 104, the connection 112 may have multiple branches, and there may be multiple mist conduits 114.

The enclosure 110 may be any space that requires sterilisation. It may be a box or room (e.g. operating theatre or hospital suite) or a vehicle interior (e.g. an ambulance or the like). The flow rate from the apparatus into the enclosure 110 may be adjustable, e.g. to facilitate the spread of hydroxyl radicals within the enclosed volume.

The sterilisation apparatus 100 further comprises a manifold 116 that is configured to combine the microwave energy, mist and gas to generate the flow 108 of hydroxyl radicals. In this embodiment, the manifold 116 defines an internal volume that operates as a plasma generating region 124 in a manner discussed in more detail below.

The manifold 116 comprises a proximal manifold inlet 118 constituted by a plurality of openings which are connected to the mist conduits 114 and a manifold outlet 120 constituted by an aperture through which the flow 108 of hydroxyl radicals passes into the enclosure 110. The manifold inlets 118 feed into the plasma generating region 124. The manifold outlet 120 is an exit aperture of the plasma generating region 124. The openings of the manifold inlet 118 may be aligned with the aperture of the manifold outlet 120 in the sense that the flow of mist from the mist conduits 114 enters the manifold 116 in a direction that is aligned with, e.g. parallel to, the direction in the which the flow 108 of hydroxyl radicals exits the manifold 116.

The manifold 116 has a body which defines a plasma cavity 126 (which is shown in FIG. 2 without the surrounding body, for clarity). The plasma generating region 124 is a volume in the plasma cavity 126 in which the plasma is generated.

The manifold 116 further comprises a plurality of lateral ports 122 that are disposed adjacent to the plasma generating region 124. In this example, the lateral ports 122 are evenly distributed around a circumference of the plasma cavity 126 (see FIG. 2). The manifold 116 may include a tube which defines the plasma cavity 126. The lateral ports 122 are arranged at the outer side surface of the tube. The axial direction of the tube defines the flow of water mist.

A power distribution device 128 is provided which distributes the microwave radiation generated by the microwave source 102 to the lateral ports 122. The power distribution device 128 includes an input port 130, a power splitter 132 and a plurality of output ports 134. The power splitter 132 is connected to the microwave source 102 via the input port 130. In the embodiment depicted in FIG. 1, only one power transmission device 128 is present. In particular, only one power splitter 132 is provided. The power splitter 132 has only one input port 130 with which the power splitter 132 is directly coupled to the microwave source 102, in particular to a source outlet of the microwave source 102.

The power splitter 132 is connected to the lateral ports 122 via the output ports 134. The input port 130, the power splitter 132 and/or the output ports 134 are constituted by waveguides 136. Thus, the power distribution device 128 supplies microwave radiation from the microwave source 102 to the plurality of lateral ports 122.

In the embodiment of FIGS. 1 and 2, the power splitter 132 is a ring coupler which includes a ring transmission line 138 and a plurality of radial transmission lines 140. The ring transmission line 138 has the form of a closed ring such that the waveguide 136 corresponding to the ring transmission line 138 has a circular passage. The radial transmission lines 140 extend from the ring transmission line 138 in a radial direction. The radial transmission lines 140 constitute the input port 130 and the output ports 134.

In embodiments depicted in FIGS. 1 and 2, the radial transmission line 140 defining the input port 130 extends radially outward while the radial transmission lines 140 defining the output ports 134 extend radially inwards.

The radial transmission lines 140 defining the output ports 134 are spaced apart in the direction of extension of the ring transmission line 138 by a multiple of half the wavelength of the microwave energy.

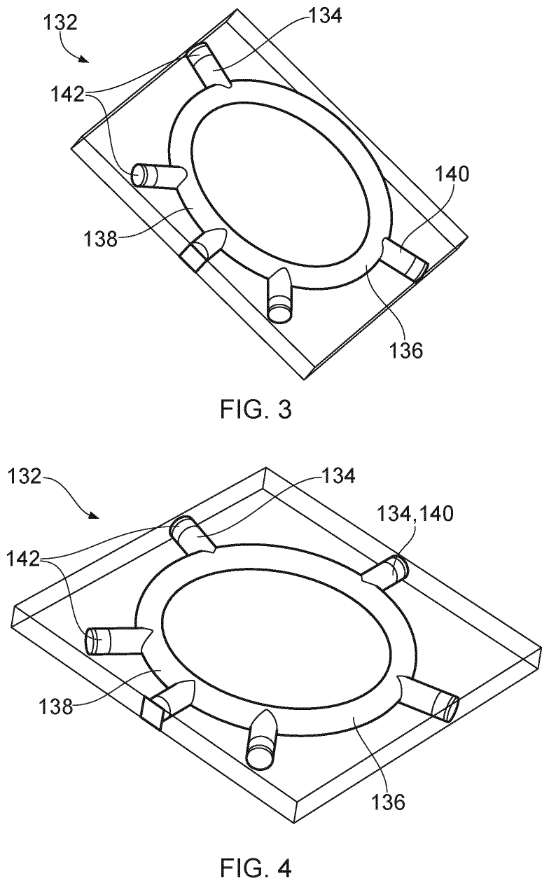

In the embodiment depicted in FIG. 3, the radial transmission lines 140 defining the output ports 134 are spaced apart by λ/2 except of those two output ports 134 which are furthest apart from the input port 130. In the embodiment depicted in FIG. 4, the radial transmission lines 140 are equally spaced apart around the ring transmission line 138 by λ/2.

The radial transmission line 140 defining the input port 130 is arranged exactly in the middle between two radial transmission lines 140 defining the output ports 134. Thus, the distances between the radial transmission line 140 defining the input port 130 along the extension of the ring transmission line 138 to the adjacent radial transmission lines 140 defining the output ports 134 corresponds to a quarter of the wavelength of the microwave energy.

The ring transmission line 138 and the radial transmission lines 140 depicted in FIGS. 1 to 4 are waveguides 136. The output ports 134 of the embodiment depicted in FIGS. 1 and 2 are not provided with adapters 142. Thus, microwave radiation is fed into the plasma generating region 124.

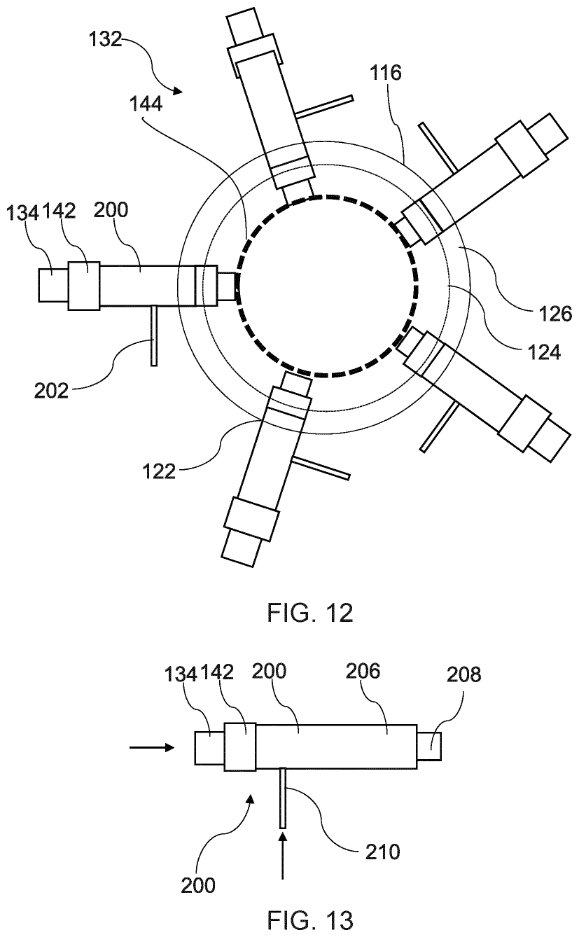

However, it is possible that the adapters 142 are provided at the output ports 134. For example, the power splitters 132 depicted in FIGS. 3 and 4 have the adapters 142 to which plasma applicators 200 can be connected to (the plasma applicators 200 are described later, see also FIG. 12).

The plasma cavity 126 and, thus, the plasma generating region 124 may be arranged in the middle of the ring transmission line 138. Such a configuration helps to homogeneously feed microwave energy into the plasma generating region 124. In addition, coaxial cables for transmitting the microwave energy from the microwave source 102 to the plasma generating region 124 may be omitted since the output ports 134 of the power splitter 132 can be directly connected to the plasma applicators 200 or, in case the plasma applicators 200 are omitted, the microwave radiation emitted at the output ports 134 can be directly fed into the plasma generating region 124.

In use, gas is supplied through the connection 112 to the mist generator 104. Mist is created by the mist generator 104 and entrained in the gas from the connection 112, whereupon it flows though the mist conduits 114 into the manifold 116. Microwave energy supplied from the microwave source 102 creates an electric field within the plasma generating region 124 to strike and/or maintain a plasma in the gas. The lateral ports 122 and, thus, the output ports 134 (including or excluding the plasma applicators 200) may be disposed around the plasma generating region 124 in a manner that ring-like plasma arc 144 is visible in the manifold outlet 120 (see in FIG. 12).

The striking of the plasma may be achieved in that the impedance of the water mist in the plasma generating region 124 is so high that a sufficiently high electrical field can be built up in the plasma generating region 124 in order to strike the plasma. The impedance of the plasma may be similar to the line impedance of the power distribution device 128 which has a line impedance for example of 50Ω.

It is also possible that the microwave radiation provided by the microwave source 102 is solely used to maintain the plasma in the plasma generating region 124. The plasma may be struck by a radiofrequency (RF) pulse which may be generated by a radio frequency generator (not shown in the figures) whose output is fed into the plasma generating region 124.

FIGS. 3 and 4 show further embodiments of the power splitter 132 of the ring coupler type. The power splitter 132 depicted in FIGS. 3 and 4 has the same characteristics as the power splitter 132 depicted in FIGS. 1 and 2 except for the following differences. All radial transmission lines 140 extend radially outwards from the ring transmission line 138. This means, the input port 130 as well as all output ports 134 extend radially outward from the ring transmission line 138. In the embodiment shown in FIGS. 3 and 4, the output ports 134 are all provided with adapters 142.

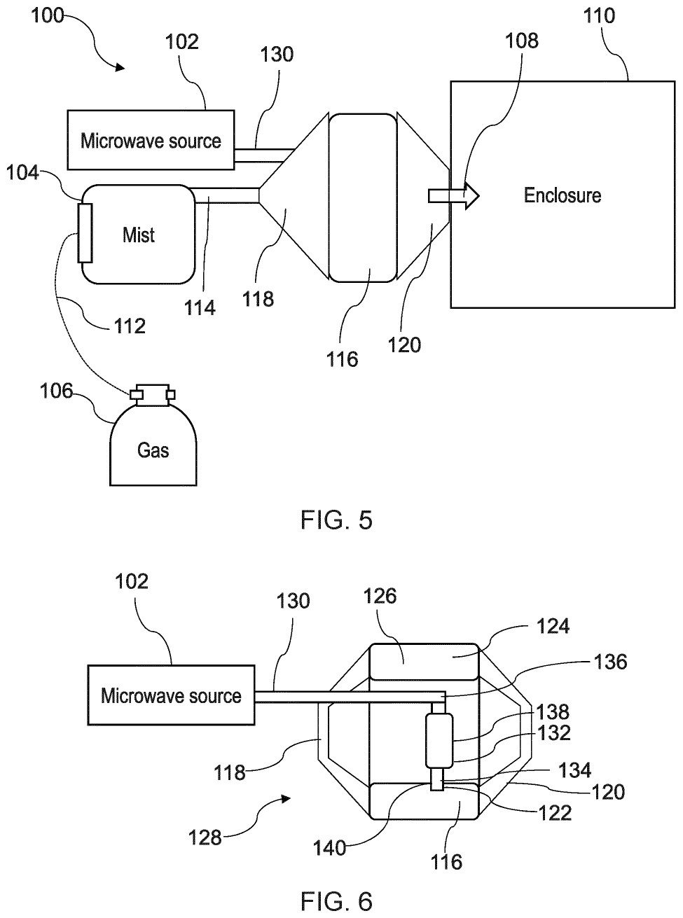

FIGS. 5 and 6 show an embodiment of the sterilisation apparatus 100 in which the ring coupler depicted in FIGS. 3 and 4 may be employed. The sterilisation apparatus 100 depicted in FIGS. 5 and 6 has the same characteristics as the sterilisation apparatus 100 depicted in FIGS. 1 and 2 except for the following differences.

The plasma cavity 126 has a toroid shape and surrounds the power splitter 132. In particular, the plasma cavity 126 is coaxially arranged to the ring transmission line 138. This means that the plasma cavity 126 and, thus, the plasma generating region 124 surround the power splitter 132.

The manifold inlet 118 may have the outer shape of a funnel and is configured to distribute the incoming flow of water mist at the connection 112 to the toroid shape of the plasma cavity 126. Similarly, the manifold outlet 120 may also have the outer shape of a funnel and is configured to channel the flow of water mist who has passed the plasma generating region 124 to a single aperture which is connected to the enclosure 110.

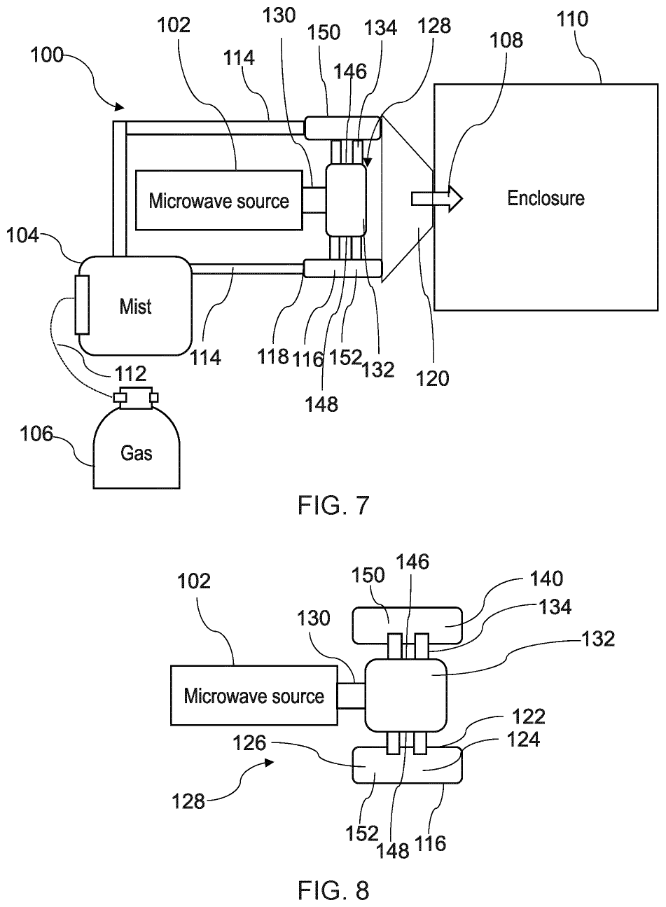

FIGS. 7 and 8 show another embodiment of the sterilisation apparatus 100. The sterilisation apparatus 100 depicted in FIGS. 7 and 8 has the same characteristics as the sterilisation apparatus 100 depicted in FIGS. 1 and 2 except for the following differences.

The power splitter 132 depicted in FIGS. 7 and 8 has its output ports 134 on the upper side 146 and a lower side 148. The output ports 134 arranged on the upper side 146 are connected to a first portion 150 of the manifold 116. The output ports 134 arranged on the lower side 148 are connected to a second portion 152 of the manifold 116. The first portion 150 and the second portion 152 each define a plasma cavity 126 such that two plasma generating regions 124 are present with the manifold 116 in this embodiment.

The first portion 150 and the second portion 152 may not be in fluid communication with each other. The first portion 150 and the second portion 152 each include lateral ports 122 to which the output ports 134 of the power splitter 132 are connected to. The first portion 150 and the second portion 152 may each have an opening for the connection 112. These openings define the manifold inlet 118. The connection 112 may include two lines which connect the mist generator 104 to the first portion 150 and the second portion 152, respectively. The manifold outlet 120 may be similar to the one depicted in FIGS. 5 and 6.

The power distribution device 128 and, in particular, the power splitter 132 may be arranged between the first portion 150 and the second portion 152.

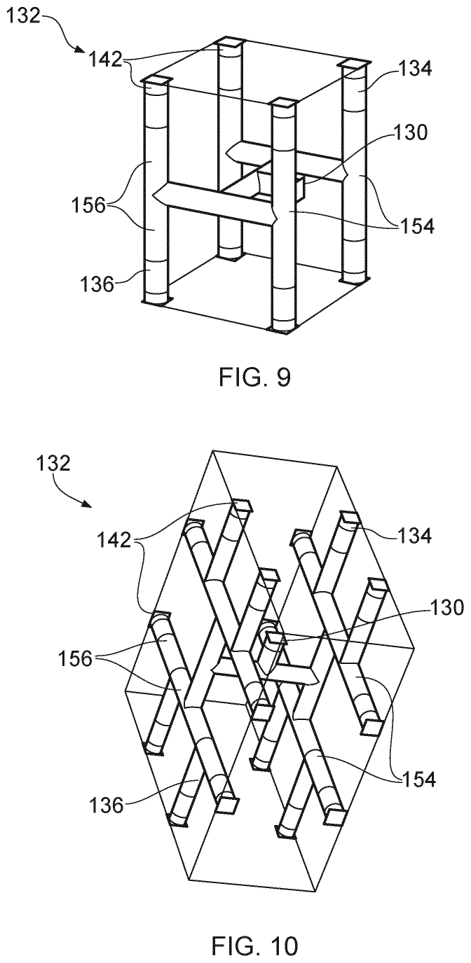

The power splitter 132 used for the sterilisation apparatus 100 as depicted in FIGS. 7 and 8 may have the configuration as depicted in FIG. 9. The power splitter 132 is an assembly of interconnected waveguides 136.

In FIG. 9, the power splitter 132 includes a plurality of waveguides 136 which is an example of an assembly of waveguides 136. The input port 130 in form of a waveguide 136 leads to another waveguide 136 at a junction 154. Thus, the microwave radiation supplied to the input port 130 is divided at the junction 154. The ratio of the division of the power of the microwave radiation at the junction 154 depends on the length of the waveguide 136 constituting the input port 130 and the lengths of waveguide sections 156 starting from the junction 154. For example, the length of the waveguide 136 constituting the input port 130 and the above described waveguide sections 156 may be a multiple of the half the wavelength of the microwave radiation.

The waveguide sections 156 each lead to another junction 154 from which two further waveguide section 156 originate. Each of the four waveguide sections 156 again leads to four junctions 154 from which two further waveguide sections 156 originate. These eight waveguide sections 156 constitute eight output ports 134 which each can be provided with adapters 142 for converting the microwave radiation into electrical energy to be supplied to the plasma applicators 200. Thus, the power distribution device 128 has one input port 130 and eight output ports 134. Four output ports 134 are arranged on the upper side 146 and four output ports 134 are arranged on the lower side 148.

Each waveguide 136 is a hollow passage for transmitting microwave radiation. The waveguides 136 and the length of the respective waveguide sections 156 is preferably adapted for an optimum transmission and for an even distribution of the power to the output ports 134. In particular, the power of the microwave radiation may be equally divided at each junction 154 such that the power of the microwave radiation present at each output port 134 is the same.

The waveguides 136 may be straight in the embodiment depicted in FIGS. 9 and 10. The waveguide 136 may be formed by drilling a hole in a block of conductive material. The block is schematically outlined in FIGS. 9 and 10 but not depicted in order to visualise the waveguides 136. This way of manufacturing the waveguides 136 is simple while ensuring a precise lengths of the waveguides 136.

In an alternative embodiment, the waveguides 136 may have a waveguide body made from a plastic material whose inner surface is covered by a conductive layer (not visible in the figures). The conductive layer ensures the transmission of the microwave radiation and has a thickness greater than the Skin-depth of the microwave radiation to be transmitted within the waveguides 136. This type of waveguide 136 is lightweight.

The embodiment of the power splitter 132 depicted in FIG. 10 has an additional layer of junctions 154 and waveguide sections 156 resulting in 16 output ports 134. Thus, this embodiment of the power splitter 132 distributes the power received at the single input port 130 to the 16 output ports 134. In particular, the power of the microwave radiation at the 16 output ports 134 is the same. The other comments and descriptions made in conjunction with the embodiment of FIG. 9 equally apply to the embodiment depicted in FIG. 10.

Thus, the power splitters 132 depicted in FIGS. 9 and 10 have a tree-like structure in which the power is input at the input port 130 and branched into two waveguide sections 156 at the junction 154. This splitting or branching of the waveguide 136 connected to the input port 130 is repeated at each further waveguide section 156.

The power splitter device 132 depicted in FIG. 10 has some output ports 134 arranged on other sides except the upper side 146 and a lower side 148; i.e. on two additional sides perpendicular to the flow of the water mist. These output ports 134 may be connected to a third portion and fourth portion of the manifold 116 (not depicted in the figures). The third portion and the fourth portion has the characteristics of the first portion 150 and the second portion 152, respectively. Alternatively, the power splitter 132 depicted in FIG. 10 may be connected to the manifold 116 having a toroid shape as depicted in FIGS. 1 and 2.

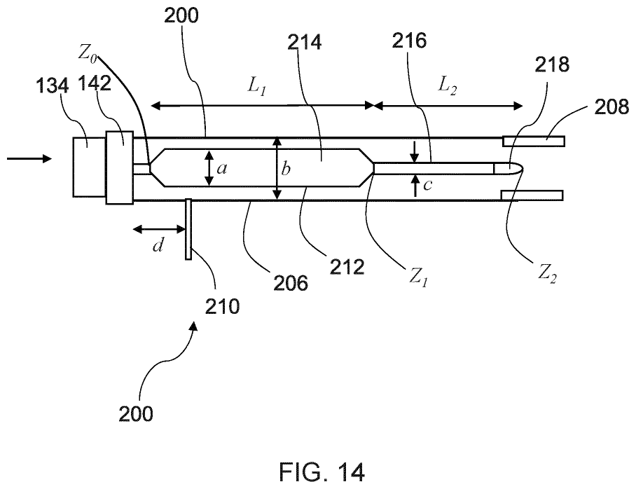

In an optional embodiment, each lateral port 122 is configured to receive a plasma applicator 200 (see for example FIG. 12). Each plasma applicator 200 is connected to receive microwave energy from the microwave source 102 via a power distribution device 128. As discussed below in more detail with reference to FIGS. 13 and 14, each plasma applicator 200 is configured to create an electric field at a distal end thereof that is capable of striking a plasma in the gas that flows through the manifold 116. Each plasma applicator 200 is positioned at its respective lateral port 122 so that its distal end lies within the plasma generating region 124.

In this example, the gas supply 106 further comprises a second connection (not shown in the Figs.) that provides a separate gas feed to each of the plasma applicators 200. Where there are a plurality of plasma applicators 200, the second connection may comprise a plurality of branches. With this arrangement, gas enters the plasma generating region 124 from both the mist conduits 114 and from the plasma applicators 200.

In use, gas is supplied through both the connection 112 and the second connection. Mist is created by the mist generator 104 and entrained in the gas from the connection 112, whereupon it flows though the mist conduits 114 into the manifold 116. Meanwhile gas flows from the second connection through the plasma applicators 200 to enter the plasma generating region 124. Microwave energy supplied from the microwave source 102 creates an electric field within the plasma generating region 124 to strike a plasma in the gas. The plasma applicators 200 may be disposed around the plasma generating region 124 in a manner that ring-like plasma arc is visible in the manifold outlet 120.

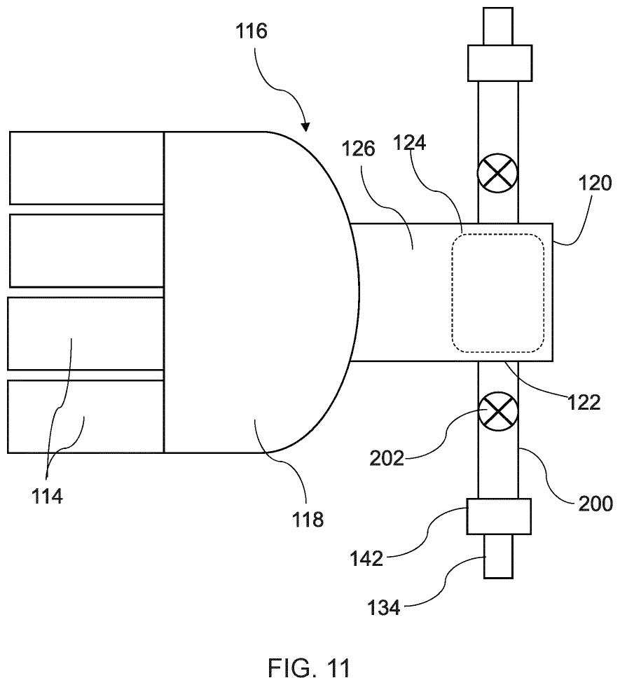

FIG. 11 is a schematic top view of a manifold 116 that can be used an embodiment of the invention. Features already discussed are provided with the same reference numbers, and description thereof is not repeated. In this example, four mist conduits 114 are received at a proximal side of the manifold inlet 118, which acts to combine the flows from each mist conduit 114 into a single tube, which extends from a distal side of the funnel element manifold inlet 118. The plasma generating region 124 is formed within the tube which defines the plasma cavity 126. The manifold outlet 120 that leads to the enclosure 110 (not shown) is at the distal end of the tube.

Similarly, the lateral ports 122 through which the plasma applicators 200 extend into the plasma generating region 124 are formed in side surfaces of the tube. Each plasma applicator 200 comprises the proximal adapter 142 that is connectable to the output port 134. As discussed above, each plasma applicator 200 has a dedicated gas feed, which enters though a gas inlet tube 202. The gas inlet tube 202 extends into a direction that is transverse to the direction in which the plasma applicator 200 extends into the plasma generating region 124. In FIG. 2 the direction of the gas inlet tube 202 is into the page.

FIG. 12 shows a front view of the manifold 116 shown in FIGS. 1 and 2. Features already discussed are provided with the same reference numbers, and description thereof is not repeated. In this example, there are five plasma applicators 200 equally distributed along the circumference of the plasma cavity 126. In this view, the portions of the plasma applicators 200 that extends into the plasma cavity 126 are visible through the manifold outlet 120. The plasma ring created in operation is shown schematically by dotted line 144. It may be seen that the flow of mist from the mist conduits 114 passes through and around the plasma ring, which thereby causes the formation of hydroxyl radicals in the gas flow to facilitate sterilisation.

FIG. 13 is a side view of a plasma applicator 200 that can be used in the apparatus discussed above. The plasma applicator 200 is a generally elongate cylindrical member, defined by a conductive tube 206, e.g. of copper or the like. The adapter 142 is mounted at a proximal end of the conductive tube 206 to be connected to the output port 134. Microwave energy conveyed along the output port 134 can therefore be delivered into the conductive tube 206 in a direction in line with a longitudinal axis of the conductive tube 206. The conductive tube 206 is open at its distal end. The gas inlet tube 202 is mounted on a side of the conductive tube 206 towards its proximal end. The gas inlet tube 202 defines a flow path that passes into an internal volume of the conductive tube 206. The flow path is angled relative to the axis of the conductive tube 206. In this example, the flow path lies transverse to that axis. Gas delivered through the gas inlet tube 202 flows through the conductive tube 206 to exit at its distal end. A quartz tube 208 in mounted coaxially with the conductive tube 206 in the distal end thereof. The quartz tube 208 protrudes beyond the distal end of the conductive tube 206, and overlaps with an inner surface of the conductive tube 206 along a distal length thereof, as shown in FIG. 14.

FIG. 14 is a schematic cross-sectional view through the plasma applicator 200 shown in FIG. 13. The plasma applicator 200 comprises an elongate conductive member 212 extending coaxially with the conductive tube 260 through the internal volume. A proximal end of the elongate conductive member 212 is connected to the inner conductor of the adapter 142. The elongate conductive member 212 has a proximal portion 214 and a distal portion 216 with differing diameters. In this example, the proximal portion 214 has a diameter a that is larger than a diameter c of the distal portion 216. The distal portion 216 terminates at a distal tip 218, which is rounded in this example. In conjunction with the conductive tube 206, the proximal portion 214 and distal portion 216 respectively define a first coaxial transmission line and a second coaxial transmission line.

The plasma applicator 200 includes a quarter wave transformer arranged to increase the impedance at the distal tip thereof to facilitate a plasma strike with delivered microwave energy. The quarter wave transformer may be provided by the first coaxial transmission line defined above, i.e. by the conductive tube 206 and proximal portion 214 of the elongate conductive member 212.

The operation of the quarter wavelength transformer is now explained. The output port 134 may have an impedance of Z₀, which may be 50Ω. An outer conductor of the adapter 142 is electrically connected to the conductive tube 206, which has a uniform inner diameter b along its length. An inner conductor of the adapter 142 is electrically connected to the elongate conductive member 212.

An impedance Z_{L}_{₁} of the first coaxial transmission line can be expressed as:

$${Z}_{{L}_{1}} = \frac{138}{\sqrt{{ε}_{r}}} {log}_{10} \frac{b}{a}$$

An impedance Z_{L}_{₂} of the second coaxial transmission line can be expressed as:

$${Z}_{{L}_{2}} = \frac{138}{\sqrt{{ε}_{r}}} {log}_{10} \frac{b}{c}$$

The first coaxial transmission line has a length L₁, and the second coaxial transmission line has a length L₂. Both L₁ and L₂ are arranged to be an odd multiple of a quarter wavelength of the microwave energy conveyed by the coaxial cable 308. For example, where the microwave energy has a frequency of 2.45 GHz, the L₁ and L₂ may be 30.6 mm, so the plasma applicator 200 itself has an overall length of 6-8 cm.

Consequently, an impedance Z₁ of the junction of the first coaxial transmission line and the second coaxial transmission line can be expressed as:

$${Z}_{1} = \frac{{( {Z}_{{L}_{1}} )}^{2}}{{Z}_{0}}$$

And an impedance Z₂ at the distal tip 218 the second coaxial transmission line can be expressed as:

$${Z}_{2} = \frac{{( {Z}_{{L}_{2}} )}^{2}}{{Z}_{1}}$$

Substituting and simplifying the above expressions permits Z₂ to be expressed as:

$$( \frac{{( {log}_{10} b - {log}_{10} c )}^{2}}{{( {log}_{10} b - {log}_{10} a )}^{2}} ) {Z}_{0}$$

For an input power P at the proximal end of the plasma applicator 200, and assuming minimal loss of energy along the first and second coaxial transmission lines, a voltage V at the distal tip may be expressed as:

V=\sqrt{PZ₂}=M\sqrt{PZ₀}

wherein M is a voltage multiplication factor equal to

$$\sqrt{\frac{{( {log}_{10} b - {log}_{10} c )}^{2}}{{( {log}_{10} b - {log}_{10} a )}^{2}}}$$

In one example, the dimensions for the plasma applicator 200 may be as follows: a=6.5 mm, b=12.5 mm, c=1 mm. This yields a voltage multiplication factor equal to 3.862. For Z₀=50Ω and an input power P=250 W, this yields a voltage at the distal tip 218 of 431.8 V. It can therefore be understood that this structure is effective in yielding a voltage that can provide an electric field at the distal end of the applicator that is high enough to cause electrical breakdown of gas conveyed through the conductive tube 206.

In FIG. 14, the gas inlet tube 202 is located at a distance d from a proximal end of the conductive tube 206. The distance d may be selected to ensure that the gas feed tube does not affect the transmission of microwave energy by the first coaxial transmission line and the second coaxial transmission line. In one example, the distance d is 15 mm.

In an embodiment not depicted in figures, the power splitter 132 includes one or more Wilkinson dividers for distributing the incoming microwave energy to a plurality of output ports 134. Wilkinson dividers may be used if the impedance of the waveguides 136 is 100Ω.

The power distribution device 128 including all its components has an impedance of 50Ω which is close to the impedance of the plasma. Thus, there is an impedance match between the plasma and the power distribution device 128 resulting in improved power transmission into the plasma.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A sterilisation apparatus comprising:
a microwave source arranged to generate microwave energy;
a mist generator arranged to generate a flow of water mist;
a manifold connected to receive the flow of water mist from the mist generator and configured to direct the flow of water mist through an internal volume thereof towards a manifold outlet; and
a power distribution device having an input port coupled to the microwave source and a plurality of output ports coupled to the internal volume of the manifold,
wherein the power distribution device is configured as a power splitter that operates to distribute microwave energy received at the input port between the plurality of output ports to generate a plasma in a plasma generating region of the internal volume.

2. The sterilisation apparatus of claim 1, wherein the power distribution device comprises an assembly of interconnected waveguides.

3. The sterilisation apparatus of claim 2, wherein each waveguide comprises a hole in a block made from a conductive material.

4. The sterilisation apparatus of claim 2, wherein each waveguide includes a waveguide body made from a plastic material, wherein the waveguide body has a passageway formed therein, an inner surface of which is covered by a conductive layer.

5. The sterilisation apparatus of claim 1, wherein the power distribution device comprises a ring coupler, wherein the plurality of output ports extend radially inwards from the ring coupler.

6. The sterilisation apparatus of claim 5, wherein a distance between adjacent output ports around the ring coupler is nλ/2, wherein n is an integer and λ is the wavelength of the microwave energy.

7. The sterilisation apparatus of claim 5, wherein the input port is disposed on the ring coupler at a location that is equidistant between a pair of output ports.

8. The sterilisation apparatus of claim 2, wherein the assembly of interconnected waveguides comprises a plurality of interconnected straight waveguides that provide a plurality of paths from the input port to the outlet ports, where each path comprises a plurality of orthogonally disposed waveguide sections that interconnect junctions between the waveguides.

9. The sterilisation apparatus of claim 1, wherein the power distribution device comprises a Wilkinson power divider.

10. The sterilisation apparatus of claim 1 further comprises a plurality of plasma applicators, which each plasma applicator is connected to a respective output port.

11. The sterilisation apparatus of claim 10, further comprising an adapter for connecting the output port to the plasma applicator.

12. The sterilisation apparatus of claim 1, wherein the plurality of output ports is disposed around the plasma generating region.

13. The sterilisation apparatus of claim 1, wherein the plasma generating region has a torus shape.

14. The sterilisation apparatus of claim 1, wherein the manifold comprises a first portion defining a first plasma generating region and a second portion defining a second plasma generating region, wherein preferably the power distribution device is arranged between the first portion and the second portion.

15. The sterilisation apparatus of claim 1, comprising a gas supply which is connected to deliver a gas flow to the mist generator, wherein preferably the gas flow entrains water mist formed by the mist generator to create the flow of water mist.

16. The sterilisation apparatus of claim 10, wherein each plasma applicator comprises:
a conductive tube; and
an elongate conductive member extending along a longitudinal axis of the conductive tube,
wherein the conductive tube and elongate conductive member provide a first coaxial transmission line at a proximal end of the plasma applicator, and a second coaxial transmission line at a distal end of the plasma applicator, and
wherein the first coaxial transmission line is configured as a quarter wavelength impedance transformer.

17. The sterilisation apparatus of claim 16, wherein the second coaxial transmission line is configured with a higher impedance than the first coaxial transmission line.

18. The sterilisation apparatus of claim 16, wherein the flow of gas received by each plasma applicator passes between the conductive tube and elongate conductive member.

19. The sterilisation apparatus of claim 16, wherein each plasma applicator comprises a gas inlet tube configured to deliver the flow of gas to a space between the conductive tube and the elongate conductive member, wherein the gas inlet tube extends transversely to the longitudinal axis of the conductive tube.

20. The sterilisation apparatus of claim 1, wherein the microwave source comprises a magnetron.

21. The sterilisation apparatus of claim 1, wherein a manifold outlet of the manifold is couplable to an enclosure that defines a space to be sterilised.
